(19) United States
(12) Patent Application Publication
(10) Pub. No.: US 2006/0134647 A1
(43) Pub. Date: Jun. 22, 2006

(54) Method for elimination of false reactivities in immunoassay for Hepatitis A virus

(76) Inventors: Sylwia Karwowska, Dobbs Ferry, NY (US); Robert C. Payne, New Fairfield, CT (US); Michael J. Sommer, Monroe, NY (US)

Correspondence Address: BAYER HEALTHCARE, LLC;LAW & PATENTS DEPT, 511 BENEDICT AVENUE, TARRYTOWN, NY, 10591-5097, US

(21) Appl. No.: 11/016,224

(22) Filed: Dec. 17, 2004

(51) Int. Cl. C12Q 1/68; G01N 33/53

(52) U.S. Cl. 435/6; 435/7.1

(57) ABSTRACT

An ancillary reducing reagent composition is used to significantly reduce and substantially eliminate the incidence of false positive results in an immunoassay for the detection of antibodies to Hepatitis A virus. The reducing agent contained in the ancillary reducing reagent composition is a sulfhydryl compound buffered to a pH sufficient to prevent oxidation of the reducing agent.

### BACKGROUND OF THE INVENTION

Hepatitis A virus is a single-stranded RNA picornavirus. The prinicpal antigenic component, HAV antigen, has been detected on the virus with polyclonal antibodies, and with monoclonal antibodies. The antibody to HAV is abbreviated anti-HAV. The detection of antibody to HAV antigen is used to monitor the progress of Hepatitis A infections.

Sandwich, competitive, ELISA and radioimmunoassays have been developed to measure the incidence of HAV specific antibodies in biological samples of serum or plasma.

### BRIEF DESCRIPTION OF THE DRAWINGS

FIG. 1 is a diagram showing the competitive assay format for the detection of antibodies specific to the HAV antigen;

FIG. 2 is a graph comparing cysteine stability at various pHs in ancillary reagent at 30° C.;

FIG. 3 is a graph comparing cysteine stability at various pHs in ancillary reagent at 40° C.;

FIG. 4 is a graph comparing cysteine stability at various pHs in ancillary reagent at 50° C.;

FIG. 5 is a bar graph showing the incidence of false positive results with and without cysteine pretreatment.

### DESCRIPTION OF THE PREFERRED EMBODIMENTS

This invention significantly reduces and substantially eliminates the occurrence of false positive results in an immunoassay for the detection of antibodies to Hepatitis A virus in immunoassays such as sandwich, competitive, ELISA and radioimmunoassays.

The Advia Centaur HAV Total Assay (Bayer HealthCare LLC) competitive immunoassay is considered the best mode for this invention, and is well known to those skilled in the art. It is an automated chemiluminometric competitive immunoassay that can be used for the detection of total antibodies to Hepatitis A virus in a biological sample, such as human serum or plasma.

In this competitive immunoassay, anti-HAV antibody in a biological sample competes with a constant amount of anti-HAV antibody labeled with a chemiluminescent acridinium ester that produces a signal for HAV antigen from anti-HAV reagents. The proportion of bound labeled anti-HAV is inversely proportional to the concentration of anti-HAV antibody in the biological sample.

It has been found that by treating test samples of human serum or plasma with a reducing agent, the incidence of false positive results from the competitive immunoassay is significantly reduced and substantially eliminated.

The reducing agent can be a sulfhydryl compound, such as 2-mercaptoethanol, dithiothreitol, dithioerythritol, cysteine, and the like. The preferred reducing agent is cysteine.

A preferred means for introducing the reducing agent in the test sample is with the use of an ancillary reagent containing the reducing agent. The concentration of cysteine in the ancillary reagent is in an amount sufficient to significantly reduce and substantially eliminate the occurrence of false positive results in an immunoassay for the detection of antibodies to Hepatitis A virus. The concentration of reducing agent in the ancillary reagent generally varies from about 140 mM to about 180 mM.

The preferred reducing agent, cysteine, is more stable in an acidic environment, for example, a pH of about 2.0 to about 7.0. In a higher pH environment above 7.0, cysteine can still reduce the incidence of false positive results in a competitive immunoassay to measure HAV specific antibodies. However, at the higher pH environment above 7.0, cysteine is not as stable for extended periods of time as it would be in the lower pH environment of about 2.0 to about 7.0.

This is an important factor because if the cysteine is oxidized to its dimer cystine, it will precipitate from the solution and not be available to reduce the incidence of false positive results when testing patient samples for Hepatitis A virus.

Accordingly, the ancillary reagent comprises a reducing agent, preferably cysteine buffered to a pH of about 2.0 to about 7.0, and preferably to a pH of about 2.5 to about 5, which most effectively prevents the oxidation of cysteine to cystine.

The buffering agent for the ancillary reagent can be a phosphate such as sodium or potassium phosphate, an acetate such as sodium acetate, acetic acid, formic acid, succinic acid, a citrate such as sodium citrate, and the like. The concentration of buffering agent in the ancillary reagent generally varies from about 8 mM to about 12 mM.

Alternatively, cysteine can be packaged in a solid form and reconstituted with a liquid reagent consisting of a buffer in deionized water. Alternatively, the buffer can be included in solid form with the cysteine and reconstituted with deionized water before use as a reducing agent. However, as already noted, the reconstituted stability would be poor if the final pH is above 7.0. Therefore, the solid cysteine should be reconstituted with a buffer at a pH of about 2.0 to about 7.0, preferably at a pH of about 2.5 to about 5.0.

The ancillary reagent can optionally include a salt such as sodium chloride or potassium chloride, in a concentration sufficient to provide a more compatible physiological environment for the antibodies in the test sample. The salt concentration can generally vary from about 450 mM to about 550 mM in the ancillary reagent.

The ancillary reagent can also optionally include a chelating agent such as ethylenediaminetetraacetate acid (“EDTA”), ethylene glycol-bis-(2-aminoethyl)-N,N,N′N′-tetraacetic acid (“EGTA”), a citrate such as sodium citrate, citric acid, salicylic acid, triethanolamine, N-(2-hydroxyethyl)ethylenedinitrilotriacetic acid (“HEDTA”), and the like, in a concentration sufficient to remove interfering substances, such as iron, copper, or zinc, which can contaminate the reagents used in the immunoassay systems, such as the Advia Centaur HAV Total Assay (Bayer HealthCare LLC). The chelating agent can generally have a concentration of about 0.9 mM to about 1.1 mM in the ancillary reagent.

The ancillary reagent can also optionally include a preservative, such as sodium benzoate, thiazolones, bromo-nitro-dioxane, methyl and propyl parabenzoic acids, and the like, in an amount sufficient to prevent the growth of microorganisms in the ancillary reagent during storage. The concentration of preservative in the ancillary reagent can generally vary from about 10 mM to about 18 mM.

In accordance with the present invention, a biological test sample of human serum or plasma is pretreated with a sufficient amount of the ancillary reagent. The biological test sample/ancillary reagent is incubated at about 20° C. to about 45° C., preferably about 30° C. to about 40° C., and most preferably about 37° C. for about 2 minutes to about 45 minutes, preferably about 3 minutes to about 20 minutes, and most preferably about 4 minutes to about 10 minutes.

The Hepatitis A antigen, HAV, is then added to the biological sample pretreated with the ancillary reagent. If the test sample contains antibodies such as IgG, IgA, and IgM that are HAV specific, they will bind to the HAV antigen, and form an immunocomplex.

A sufficient amount of chemiluminescent reagent and a sufficient amount of solid phase reagent are then added to the biological sample/ancillary reagent to form a reaction mixture. The cysteine concentration in the reaction mixture of sample/ancillary reagent is sufficient to significantly reduce and substantially eliminate the occurrence of false positive results in the immunoassay for the detection of antibodies to hepatitis A virus.

The chemiluminescent reagent comprises a mouse monoclonal antibody tagged with a chemiluminescent acridinium ester specific for the HAV antigen. The chemiluminescent reagent then attaches to any unbound antigen.

The solid phase reagent comprises a biotinylated Fab fragment of an HAV antigen specific mouse monoclonal antibody bound to a streptavidin coated magnetic particle.

The final concentration of reducing agent in the reaction mixture is about 15 mM to about 25 mM, and preferably, about 20 mM.

The final concentration of buffering agent in the reaction mixture is about 0.9 mM to about 1.3 mM, and preferably about 1.1 mM.

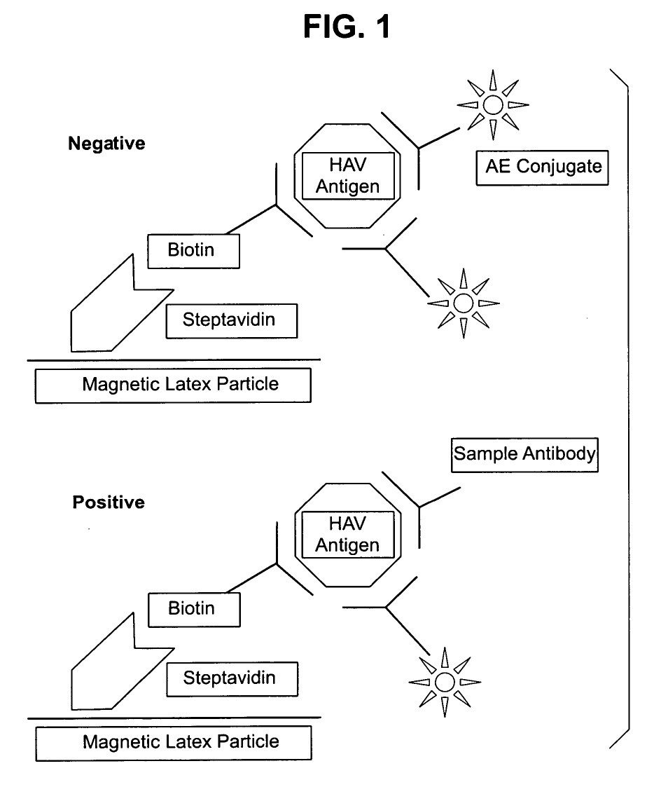

FIG. 1 is a diagrammatic representation of the competitive assay format for the detection of antibodies specific to the HAV antigen using an automated diagnostic analyzer such as the Advia Centaur HAV Total Assay (Bayer HealthCare LLC).

Antibody that is specific for the HAV antigen and tagged with the chemiluminescent acridinium ester together with the solid phase reagent is added to the sample. The chemiluminescent reagent conjugate with the mouse monoclonal antibody specific for the HAV antigen competes with HAV specific antibodies present in the test sample.

The solid phase reagent binds to the antigen/antibody/chemiluminescent reagent complexes and magnetically removes the immunocomplexes from the sample matrix by washing with a buffer. The remaining sample matrix comprises all other components in serum or plasma except antibodies, such as IgG, IgM, IgA that are specific for the HAV antigen, and have formed the immunocomplex.

The washed antigen/antibody/chemiluminescent reagent immunocomplexes are then re-suspended in an acidic reagent, such as nitric acid, typically 0.1N nitric acid, followed by contacting with a basic reagent, typically 0.25N sodium hydroxide, to thereby initiate the chemiluminescent reaction that enables measurement of the amount of antibody in relation to HAV present based on the amount of relative light units detected from the chemiluminescent reaction.

If there is a high titer of HAV specific antibodies in the sample, it will compete with the acridinium ester labeled antibody for the added antigen and a reduction in signal will occur. In contrast, if the titer of HAV specific antibody is low, then more acridinium ester labeled antibody will attach to the added antigen resulting in high signal output. Thus, the concentration of HAV specific antibody in the biological test sample is inversely proportional to the intensity of signal output from the chemiluminescent reaction.

Therefore, in a competitive assay format, a high signal from the chemiluminescent reaction indicates a low titer of HAV specific antibody in the sample and a low signal from the chemiluminescent reaction would indicate a high titer of HAV specific antibody in the sample.

In the example which follows, all concentrations are based on molarity unless otherwise noted.

### EXAMPLE 1

### Ancillary Reagent Formulation

TABLE 1

| Component | Concentration | |
|---|---|---|
| Cysteine | 170 | mM |
| Sodium Citrate | 10 | mM |
| Sodium Chloride | 500 | mM |
| Ethylenediaminetetraacetic Acid (EDTA) | 1.0 | mM |
| Sodium Benzoate | 14 | mM |

Several duplicate aliquots of ancillary reagent having the composition shown in Table 1 were prepared at a pH of 3.0, a pH of 4.0 and a pH of 5.0, respectively.

One group of reagents at a pH of 3.0, a pH of 4.0 and a pH of 5.0 was stressed at a temperature of 30° C. for periods of 3 days, 5 days and 8 days. A second group of reagents at pH's of 3.0, 4.0 and 5.0 were stressed at a temperature of 40° C. for periods of 3 days, 5 days and 8 days. Lastly, a third group of reagents at pH's of 3.0, 4.0 and 5.0 were stressed at a temperature of 50° C. for periods of 3 days, 5 days and 8 days.

The cysteine concentration in each group of reagents was then measured using Ellman's Reagent.

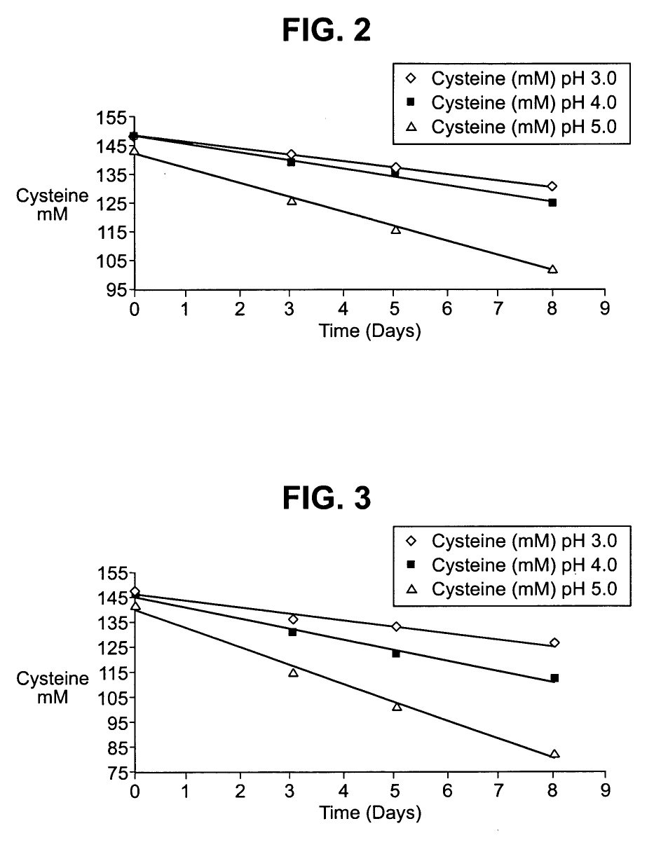

FIG. 2 is a graph showing cysteine stability in the ancillary reagent composition of Table 1 at pHs of 3.0, 4.0 and 5.0 after each of these compositions were stressed at a temperature of 30° C. for 3 days, 5 days and 8 days.

FIG. 3 is a graph showing cysteine stability in the same ancillary reagent composition of Table 1 at pH's of 3.0, 4.0, and 5.0, stressed to a temperature of 40° C. for 3 days, 5 days and 8 days.

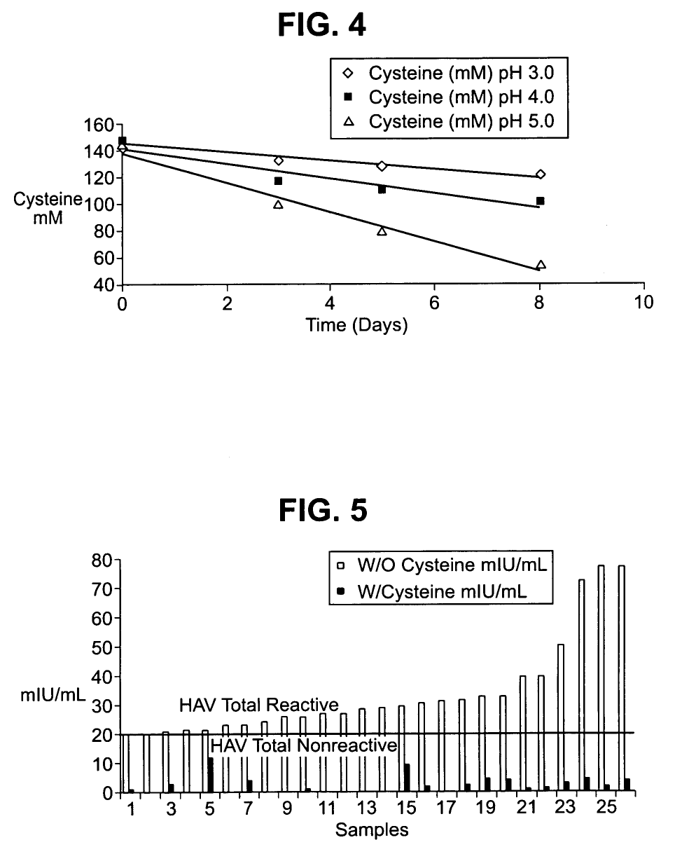

FIG. 4 is a graph showing cysteine stability in the same ancillary reagent composition of Table 1 at pH's of 3.0, 4.0, and 5.0, stressed to a temperature of 50° C. for 3 days, 5 days and 8 days.

The information shown in FIGS. 2, 3 and 4 demonstrates that lowering the pH from 5.0 to 3.0 significantly improves the stability of cysteine in the HAV total ancillary reagent. This is indicated by a higher cysteine concentration at lower pH, regardless of the stress temperatures. Consequently, reducing the pH appears to provide a proton-rich medium to maintain the sulfhydryl group on the cysteine molecules, thereby retarding the oxidative formation of cystine.

Additionally, no precipitate was observed in the ancillary reagent adjusted to a pH of 3.0 at any of the stress temperatures during the test periods. These results suggest that any cystine that forms remains soluble in the acidic reagent matrix.

FIG. 5 is a bar graph showing the incidence of false positive results for samples of HAV total reagent before and after pretreatment with ancillary reagent containing cysteine.

FIG. 5 demonstrates the incidence of HAV total false positives is quite high for the test samples without cysteine. When cysteine was added to the test samples, the occurrence of false positive results was significantly reduced. The black horizontal reference line in FIG. 5 is equal to 20 mIU/ml and represents the cutoff point in measuring the effectiveness of the Advia Centaur HAV Total Assay (Bayer HealthCare LLC).

Biological samples that yielded a test value above the cutoff line were positive for Hepatitis A. Biological samples that showed test values below the cutoff line were negative for Hepatitis A.

What is claimed is:

1. A method for reducing the occurrence of false positive results in an immunoassay for the detection of antibodies for Hepatitis A virus, comprising contacting a biological sample of serum or plasma being tested in a reaction mixture for the presence of antibodies to Hepatitis A virus with a sufficient amount of a reducing agent adapted to prevent the occurrence of false positive results.

2. The method of claim 1, wherein the concentration of reducing agent in the reaction mixture varies from about 15 mM to about 25 mM.

3. The method of claim 1, wherein the reducing agent is a sulfhydral compound.

4. The method of claim 3, wherein the sulfhydral compound is selected from the group consisting of 2-mercaptoethanol, dithiothreitol, dithioerythritol, cysteine, and mixtures thereof.

5. The method of claim 4, wherein the reducing agent is cysteine.

6. The method of claim 1, wherein the reducing agent is contained in an ancillary reagent solution buffered with a buffering agent to a pH of about 2.0 to about 7.0 to prevent oxidation of the reducing agent.

7. The method of claim 6, wherein the reducing agent is cysteine.

8. The method of claim 6, wherein the concentration of reducing agent in the ancillary reagent varies from about 140 mM to about 180 mM.

9. The method of claim 6, wherein the buffering agent is selected from the group consisting of a phosphate, an acetate, acetic acid, formic acid, succinic acid, and a citrate.

10. The method of claim 9, wherein the buffering agent is sodium citrate.

11. The method of claim 6, wherein the concentration of buffering agent in the ancillary reagent varies from about 8mM to about 12 mM.

12. The method of claim 6, wherein the concentration of buffering agent in the reaction mixture varies from about 0.9 mM to about 1.3 mM.

13. The method of claim 6, wherein the ancillary reagent includes NaCl in a concentration sufficient to provide a more compatible physiological environment for the antibodies in the biological test sample.

14. The method of claim 6, wherein the ancillary reagent includes a chelating agent in a concentration sufficient to assist removal of potential interfering substances.

15. The method of claim 6, wherein the ancillary reagent includes a preservative in a concentration sufficient to prevent the growth of microorganisms.

16. The method of claim 1, wherein the immunoassay is selected from the group consisting of competitive, sandwich, ELISA and radioimmuno assay.

17. The method of claim 16, wherein the immunoassay is competitive.

18. An ancillary reagent composition adapted to reduce the occurrence of false positive results in an immunoassay for the detection of antibodies to Hepatitis A virus, comprising a sufficient amount of a reducing agent and a sufficient amount of buffering agent to prevent oxidation of the reducing agent.

19. The ancillary reagent composition of claim 18, wherein the reducing agent is a sulfhyrdyl compound.

20. The method of claim 19, wherein the sulfhydral compound is selected from the group consisting of 2-mercaptoethanol, dithiothreitol, dithioerythritol, cysteine, and mixtures thereof.

21. The method of claim 20, wherein the reducing agent is cysteine.

22. The method of claim 18, wherein the concentration of reducing agent in the ancillary reagent varies from about 140 mM to about 180 mM.

23. The method of claim 18, wherein the buffering agent is selected from the group consisting of a phosphate, an acetate, acetic acid, formic acid, succinic acid, and a citrate.

24. The method of claim 23, wherein the buffering agent is sodium citrate.

25. The method of claim 18, wherein the concentration of buffering agent in the ancillary reagent varies from about 8mM to about 12 mM.
